SEQUENCE LISTING (9 sequences)

SEQ ID NO: 1 - protein, 330 aa, Streptococcus pyogenes
NHKVHMHHHHHHADEQEEKAKVRTELIQELAQGLGGIEKKNFPTLGDEDLDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 2 - protein, 292 aa, Streptococcus pyogenes
NHKVHMHHHHHHDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 5 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPAGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 6 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPQGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 7 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPAGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 8 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPSGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 9 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPKGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

(19) United States
(12) Patent Application Publication
(10) Pub. No.: US 2026/0242426 A1
(43) Pub. Date: Aug. 20, 2026

(54) THIOLATED RECOMBINANT BACTERIAL COLLAGEN-LIKE PROTEINS

(71) Applicant: Evonik Operations GmbH, Essen (DE)

(72) Inventors: Sven WEBER, Dossenheim (DE); Maria MONTERO MIRABET, Mühltal (DE); Ute SCHEPERS, Troisdorf (DE)

(73) Assignee: Evonik Operations GmbH, Essen (DE)

(21) Appl. No.: 19/163,267

(22) PCT Filed: Mar. 8, 2024
(86) PCT No.: PCT/EP2024/056156

§ 371 Date: Sep. 8, 2025

(30) Foreign Application Priority Data
Mar. 16, 2023 (EP) 23162203.6

(51) Int. Cl. C07K 14/315; A61L 27/22; A61L 27/52; C09D 11/04

(52) U.S. Cl. CPC C07K 14/315; A61L 27/227; A61L 27/52; C09D 11/04

(57) ABSTRACT

Recombinant bacterial collagen-like protein, preferably with an amino acid sequence that is at least ≥60% identical to the amino acid sequence of SEQ ID NO:1 wherein the amino acid sequence comprises a deletion of at least 38 amino acids at the N-terminus of the amino acid sequence of SEQ ID NO:1, wherein the recombinant collagen-like protein is functionalized with at least one thiol group. A bioink comprising the functionalized recombinant bacterial collagen-like protein, at least one solvent and optionally at least one photoinitiator, a process for producing a hydrogel by crosslinking the functionalized recombinant bacterial protein or the bioink as well as the hydrogel obtained therefrom. Furthermore, a scaffold for tissue engineering comprising the hydrogel.

### FIELD OF THE INVENTION

The invention relates to functionalized recombinant collagen-like proteins (CLPs) of bacterial origin, their application as a bioink for cell encapsulation, as well as preparation of 3D scaffolds, in vitro models and tissue grafts.

### BACKGROUND OF THE INVENTION

Bioinks which are commonly used for bioprinting, are largely synthetic thermoplastic polymers or animal-derived proteins e.g., collagen, gelatin, (meth)acryloyl functionalized animal derived collagen- or gelatin methacrylate.

Collagen-based hydrogels are optimal scaffold materials for cell encapsulation. Their biodegradability and biocompatibility allow optimal material conditions for bioprinting while a multiplicity of cell interaction domains (naturally present in collagen) allow cell spreading and scaffold colonization.

US2016/0051727 discloses a collagen based polymeric material comprising collagen molecules and/or collagen derived molecules which are functionalized by the addition of one or more ethylenically unsaturated groups and which are cross-linked via said groups.

US 2020/0179562 discloses curable recombinant human collagen functionalized with methacryloyl, which can be formulated into inks for additive manufacturing and printed into 3 dimensional objects.

US 2016/0193384 discloses the production of a hydrogel by photocrosslinking a methacryloyl or acryloyl collagen and a synthetic polymer. Such hydrogels can be used as 3D scaffolds and implants and they are suitable as inks for 3D printing in order to prepare complex 3D structures, or for incorporating cells into the structure.

Photocurable gelatin-based hydrogels have established themselves as powerful bioinks in tissue engineering due to their excellent biocompatibility, biodegradability, light responsiveness, thermosensitivity and bioprinting properties. While gelatin methacryloyl (GelMA) been the gold standard for many years, thiol-ene hydrogel systems based on norbornene-functionalized gelatin (GeINB) and a thiolated crosslinker have recently gained increasing importance (Göckler et al. 2021). Göckler et al. disclose a highly reproducible water-based synthesis of GelNB covering a broad range of degrees of functionalization (DoF: 20% to 97%) as well as its mixing with thiolated gelatin (GelS) resulting in the superfast curing photoclick hydrogel GelNB/GelS.

However, the collagens used in the above referenced prior art are obtained from human or animal sources. Such human or animal-derived collagens are not well defined due to their natural origin, are viscous and have poor aqueous solubility under physiological conditions. Due to their viscosity, significant force is needed to extrude or jet these collagen mixtures during the printing process. This results in greater shear stress and thus lower cell viability if cells are co-printed in the same mixture. The low pH-value needed to solubilize animal-derived collagen also makes it challenging to increase the concentration. A further disadvantage due to the origin is the varying quality as well as the risk of disease transmission.

Collagen-like proteins (CLPs) of bacterial origin have interesting mechanical properties, similar to those of higher eukaryotes' collagen proteins, without needing the complex maturing steps required for the eukaryotic counterparts. CLPs present a common structure: two alpha helixes, stabilizing each other, constitute a “V domain”, which is followed by a rod-like, structural collagen domain (CL). After the collagen domain, typically a membrane anchor (GPI-like) is present at the C-terminal end of the protein.

The most industrially relevant CLP is Scl2 of Streptococcus pyogenes. As described in various publications (Lukomski et al. 2002, Brodsky et al. 2009) the current understanding is that the V-domain is required for folding three Scl2 protein monomers into one triple helical structure in vitro.

Whilst bioinks based on human or animal-derived collagens are widely used, there is still an unmet need for GMP compliant bioinks to facilitate the translation of bioprinted clinical products from bench-to-bedside, particularly water-soluble biomaterials which can form hydrogels and thus support cell encapsulation and bioprinting. Required are properties such as a constant quality of produced batches, high purity as well as good solubility in conditions that provide for a suitable environment for the encapsulated cells.

It is therefore an object of the present invention to provide a protein that can be formulated into a bioink and crosslinked into a hydrogel, and which can at least reduce the disadvantages of prior art materials in particular animal derived collagens.

### SUMMARY OF THE INVENTION

The present invention therefore relates in one aspect to a recombinant bacterial collagen-like protein, preferably comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1, wherein the recombinant collagen-like protein is functionalized with at least one thiol group.

In one embodiment of the present invention the amino acid sequence is at least 60%, preferably at least 70%, more preferably at least 80%, most preferred at least 90% identical to the amino acid sequence of SEQ ID NO:2, SEQ ID NO:3 or SEQ ID NO:4.

In a further embodiment the degree of functionalization with thiol groups of the recombinant bacterial collagen-like protein ranges from 5% to 100% of the sum of primary amines comprising the N-terminal primary amine group and the primary amine groups of the lysine residues of the whole recombinant collagen-like protein.

In a further aspect the present invention relates to a bioink composition comprising
a) from about 0.125 to about 15 wt.-% of at least one functionalized recombinant bacterial collagen-like protein according to the present invention;
b) from about 75 to about 99 wt.-% of aqueous solvent;
c) from about 0 to about 2 wt.-% of at least one photoinitiator;
d) from about 0 to about 5 wt.-% of at least one photocrosslinkable polymer or other photocrosslinkable peptides;
e) from about 0 to about 15 wt.-% of at least one compound selected from additives, rheology modifiers, biopolymers, gelation enhancers, bioactive moieties, peptides, nanocellulose and/or cells;
provided that the sum of all components of the bioink amount to 100 wt.-%.

In one embodiment of present invention the bioink composition further comprises from about 0.125 to about 10 wt.-% of at least one further recombinant bacterial collagen-like protein, wherein said further recombinant bacterial collagen-like protein comprises a functionalization that is different from the functionalization of the functionalized recombinant bacterial collagen-like protein of a).

In a further embodiment of present invention the said at least one further functionalized recombinant bacterial collagen-like protein is a recombinant bacterial collagen-like protein comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1 and wherein the recombinant collagen-like protein is functionalized with at least one alkene group preferably at least one non-terminal alkene group.

In one embodiment of present invention the said at least one further functionalized recombinant bacterial collagen-like protein is a recombinant bacterial collagen-like protein comprising an amino acid sequence that is at least 60%, preferably at least 70%, more preferably at least 80%, most preferred at least 90% identical to the amino acid sequence of SEQ ID NO:2, SEQ ID NO:3 or SEQ ID NO:4.

In one embodiment of the bioink composition of present invention the degree of functionalization of the said further functionalized recombinant bacterial collagen-like protein ranges from 5% to 100% of the sum of primary amines comprising the N-terminal primary amine group and the primary amine groups of the lysine residues of the whole recombinant collagen-like protein.

In a further embodiment of the bioink composition of present invention the at least one non-terminal alkene group is selected from linear, branched or cyclic non-terminal alkenes, preferably from norbornene or its derivatives, or combinations thereof.

In a further aspect the present invention relates to a process for producing a hydrogel by crosslinking, preferably photocrosslinking the functionalized recombinant bacterial collagen-like protein or the bioink composition as described herein.

In one embodiment of the process of present invention the bioink composition is photocrosslinked using UV light, or visible light, preferably blue light.

In yet a further aspect the present invention relates to a hydrogel obtained by the process according to the present invention, preferably an injectable hydrogel.

In one embodiment of the hydrogel of present invention the hydrogel further comprises nanocellulose, peptides or mixtures thereof.

In yet a further aspect the present invention relates to a scaffold for tissue engineering comprising the hydrogel as described herein.

### FIGURES

FIG. 1: (A) Reaction scheme of the thiolation of the recombinant bacterial collagen-like protein of present invention resulting in a recombinant bacterial collagen-like protein comprising a thiol group (rColS) of present invention. (B) Subsequent disulfide bond formation of rColS for physical gelation.

FIG. 2: (A) Colorimetric Ellman assay of the functionalized recombinant bacterial collagen-like protein comprising a thiol group (rColS) of present invention in comparison to unmodified recombinant bacterial collagen-like protein (CLP). (B) DoF correlation of ¹H-NMR spectra and the Ellman Assay signal.

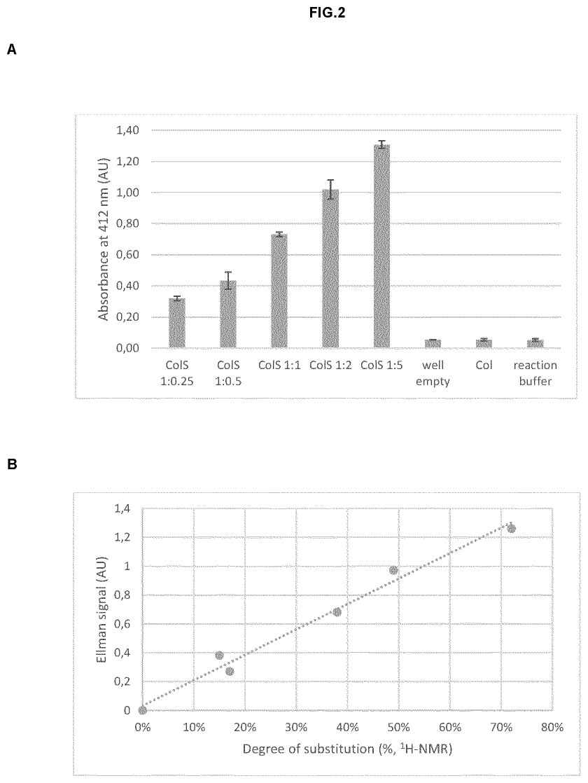

FIG. 3: ¹H-NMR example of the functionalized recombinant bacterial collagen-like protein comprising thiol groups (rColS) of present invention in comparison to unmodified recombinant bacterial collagen-like protein (CLP).

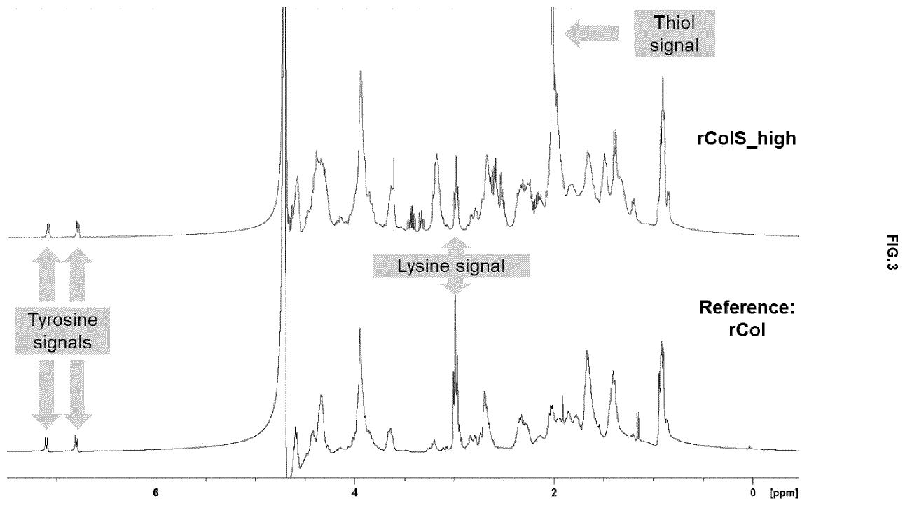

FIG. 4: 3T3 cell viability after exposure to rCol and rColS at different concentrations for 24 h. Subsequently, a CellTiter-Glo® cell proliferation assay was performed to determine the cell viability. The applied molar ratios equal the following DoFs in %: 1:0.25=15%; 1:0.5=22%; 1:1=39%; 1:2=55% and 1:5=72%.

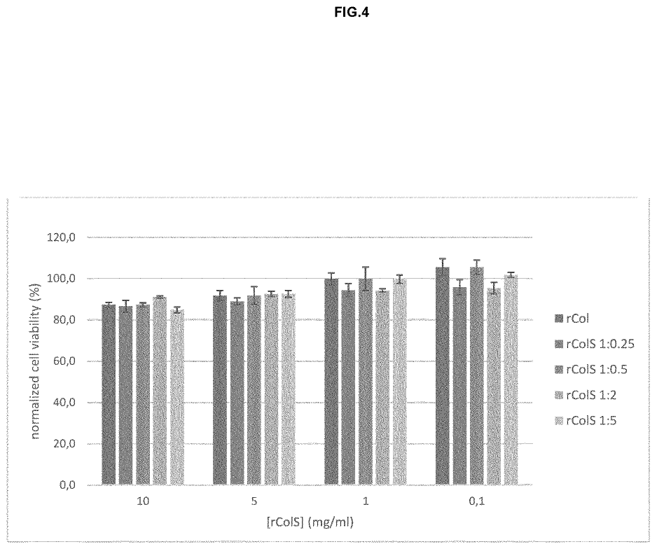

FIG. 5: Reaction scheme of the light-induced crosslinking reaction of the functionalized recombinant bacterial collagen-like protein comprising a thiol group (rColS) of the present invention and the further recombinant bacterial collagen-like protein comprising a norbornene group (rColN) of the present invention with the photoinitiator LAP (lithium phenyl-2,4,6-trimethylbenzoylphosphinate).

FIG. 6: Stained human dermal fibroblasts (HDF cells) after cultivation for 1, 7, 14 and 21 days inside of rColN/rColS hydrogel composite. A homogenous cell distribution throughout the hydrogel was observed as well as nearly 100% cell viability after LIVE/DEAD staining.

FIG. 7: A) Printed droplets (50 nl) in different shapes with rColN/rColS mixture described in the text. B) Printed droplet-based hydrogel cylinder by Drop-on-Demand of 1% rColN and 1% rColS formulations. Both materials had a DoF of ~50%+5%, 0.03% LAP was used. As solvent, 1×PBS was used. C) Stereolithographic print of a CLP cube made of 1% rColN and 1% rColS. Both materials had a DoF of ~50%±5%, 0.03% LAP was used.

FIG. 8: A) Hydrogel formulation of 100 mg/ml ColS (DoF of 16%) in different buffer systems after 24 h storage time at 4° C. f.l.t.r.: 1×PBS, 0.1 M HEPES buffer pH 8.0 and carbonate buffer pH 10. B) Incubation of 100 mg/ml ColS with a DS of (left) 16% and (right) 54% in carbonate buffer pH-value 10 overnight. Subsequently, incubation for 10 min at 37° C. liquified the formulation with DS 16% but not the formulation with DS 54%.

FIG. 9: Injectability trial with ColS hydrogel.

FIG. 10: Gelation reversibility trials with 100 mg/ml ColS (DS of 16%).

FIG. 11: Correlation between different rCol and rColS (72% DoF) concentrations dissolved in water over night and the subsequent measured viscosity using an Anton Paar MCR 502 WESP system with a 50 mm plate-cone system (1° angle; N=3). Shown data proofs the increase of viscosity due to the introduced thiol modification compared to the unmodified material. This effect is strongly concentration dependent.

FIG. 12: Example measurement shear storage modulus for the online measurement using ElastoSens Bio.

### SEQUENCES

SEQ ID NO:1 Streptococcus pyogenes Collagen-like protein (CLP), full length protein
SEQ ID NO:2 Streptococcus pyogenes CLP, truncation 3
SEQ ID NO:3 Streptococcus pyogenes CLP, truncation 5
SEQ ID NO:4 Streptococcus pyogenes CLP, no V-domain

### DETAILED DESCRIPTION OF THE INVENTION

Collagen-based hydrogels are optimal scaffold materials for cell encapsulation. Their biodegradability and biocompatibility allow optimal material conditions for bioprinting while a multiplicity of cell interaction domains (naturally present in collagen) allow cell spreading and scaffold colonization. The currently available and described collagen-based hydrogels are based on human or animal sources. Such human or animal-derived collagens have various disadvantages such as varying quality, high viscosity, poor aqueous solubility under physiological conditions as well as the potential for carrying diseases. Due to their viscosity, significant force is needed to extrude or jet the viscous collagen mixture during the printing process. This results in greater shear stress (and thus lower cell viability) if cells are co-printed in the same mixture. Furthermore, the low pH-value needed to solubilize animal-derived collagen makes it challenging to increase the concentration.

As there is a high need for optimized bioinks that overcome the above disadvantages, the inventors have set out to provide a recombinant collagen like protein that is of bacterial origin and thus a vegan alternative to the animal-derived collagen materials of the prior art and that can be functionalized and used to prepare optimized bioinks for cell-encapsulation and hydrogel production.

### Recombinant Collagen-Like Protein (CLP)

The most industrially relevant CLP of Streptococcus pyogenes Scl2 contains a large V-domain which makes up for approximately one third of the whole sequence of Scl2 and hinders the protein to be transported out of the Pichia pastoris host. This requires a complex downstream process containing cell lysis to remove the target protein from the cell. Furthermore, the V-domain itself has pathogenic properties and thus needs to be removed during the purification process by protease digestion. Usage of a protease is quite costly, and it needs to be removed during downstream processing. The production of a purified Scl2 starting from a full-length protein including the V-domain thus requires additional costly downstream process steps.

The inventors have now surprisingly found that truncated variants of the collagen-like protein, including variants with a truncated V-domain or without any V-domain lead to increased production of collagen-like protein and secretion into the fermentation medium. It was further surprising that the truncated variants were correctly folded even in absence of the V-domain.

Other than previously described in the prior art the V-domain thus does not seem to be required for the correct folding of the three Scl2 protein monomers into one triple helical structure in vitro, which opened up the possibility to overcome the various challenges caused by expressing the full lengths Scl2 protein first and removing the V-domain later.

The present invention therefore relates in one aspect to a recombinant bacterial collagen-like protein, preferably comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1, wherein the recombinant collagen-like protein is functionalized with at least one thiol group.

In one aspect it is preferred that the recombinant bacterial collagen-like protein comprises a deletion of between 38 to 90 amino acids at the N-terminus of the amino acid sequence as shown in SEQ ID NO: 1, where SEQ ID NO:1 depicts the amino acid sequence of the full-length CLP. Preferred is the deletion of between 38 to 74 amino acids. This includes a complete deletion of the N-terminal V-domain (comprising 74 amino acids) and different truncations of the V-domain of at least 38 amino acids.

The amino acid sequence of the recombinant bacterial collagen-like protein of present invention is preferably at least 60%, identical to the amino acid sequence of SEQ ID NO:2, SEQ ID NO:3 or SEQ ID NO: 4. SEQ ID NO:2 and SEQ ID NO:3 are truncated versions of Scl2 in which amino acids have been deleted as follows:
SEQ ID NO:2 is based on SEQ ID NO. 1 with a deletion of amino acids at position 13-50 (38 aa)
SEQ ID NO:3 is based on SEQ ID NO. 1 with deletion of amino acids at position 1-74 (75 aa)
SEQ ID NO:4 is based on SEQ ID NO. 1 with deletion of aa 1-90 (90 aa)

It is to be understood that the SEQ ID NOs indicated herein describe the amino acid sequence as such before functionalization with a thiol group and not the functionalized recombinant bacterial collagen-like protein.

In a preferred embodiment the recombinant bacterial collagen-like protein comprises an amino acid sequence that is at least 90%, 91%, 92%, 93%, 94%, 95%, 96% identical to the amino acid sequences of SEQ ID NO:2, SEQ ID NO:3 or SEQ ID NO:4. Preferably the recombinant bacterial collagen-like protein comprises an amino acid sequence that is at least 97%, more preferably at least 98%, most preferred at least 99% identical to the amino acid sequences of SEQ ID NO:2, SEQ ID NO: 3 or SEQ ID NO:4.

In the most preferred embodiment, the recombinant bacterial collagen-like protein consists of amino acid sequences according to SEQ ID NO:2, SEQ ID NO:3 or SEQ ID NO:4.

In a further preferred embodiment other truncated variants of the collagen-like protein from Streptococcus pyogenes are used as a basis for preparing a recombinant bacterial collagen-like protein functionalized with at least one thiol group according to present invention.

The present invention therefore also encompasses variants of the recombinant bacterial collagen-like proteins according to SEQ ID NO:1 to 4, wherein said variants preferably comprise one or more amino acid exchange(s), insertion(s) and/or deletion(s). In a preferred embodiment such variant contains up to 5, up to 4, up to 3, or up to 2, amino acid exchanges, insertions and/or deletions.

In another preferred embodiment of the present invention the variant of the recombinant bacterial collagen-like protein comprises an amino acid sequence that is at least 60%, 61%, 62%, 63%, 64%, 65%, 66%, 67%, 68%, 69%, preferably at least 70%, 71%, 72%, 73%, 74%, 75%, 76%, 77%, 78%, 79%, more preferably at least 80%, 81%, 82%, 83%, 84%, 85%, 86%, 87%, 88%, 89%, most preferred at least 90%, 91%, 92%, 93%, 94%, 95%, 96%, 97%, 98%, 99% identical to the amino acid sequence of SEQ ID NO:2, SEQ ID NO:3 or SEQ ID NO:4.

The recombinant bacterial collagen-like proteins (CLP) of present invention can be produced in a process comprising the following steps:
a) fermentation of a bacterial, yeast or plant host cell, expressing a CLP with an amino acid sequence that is at least ≥60% identical to the amino acid sequence of SEQ ID NO: 1, in a medium, wherein the amino acid sequence comprises a deletion of at least 38 amino acids at the N-terminus of the amino acid sequence of SEQ ID NO:1,
b) accumulation of the bacterial CLP in the medium, wherein a fermentation broth is obtained,
c) separating the bacterial, yeast or plant host cells from the fermentation broth,
d) incubating the fermentation broth for at least 1 h at not more than 25° C. for folding of the CLP,
e) optionally purification of the bacterial CLP by at least one of the following: solvent precipitation, tangential flow filtration (TFF), ion exchange chromatography, reversed-phase chromatography.

In a preferred embodiment the folding of CLP in step d) is performed at a temperature between-80° C. and 25° C., preferably between 0° C. and 20° C. In a preferred configuration folding is performed in presence of glycerin or salts.

In another preferred embodiment, folding of CLP in step d) is performed for a time between 1 h and 48 h, preferably between 1 h and 24 h.

In another preferred embodiment, folding of CLP in step d) is performed with a concentration of CLP of at least 1 mg/ml, preferably at least 4 mg/ml.

In a preferred embodiment, the host cell is a microorganism of the species P. pastoris, E. coli, P. putida or C. glutamicum comprising any of the polypeptides according to the present invention.

In a preferred embodiment, the microorganism is a yeast of the genus P. pastoris or a bacterial cell, preferably E. coli, Corynebacterium or Brevibactetium.

The microorganism may be a microorganism in which the nucleotide sequence encoding the CLP is present in overexpressed form.

### Functionalization

The recombinant bacterial collagen-like protein of present invention and as described herein above is functionalized with at least one thiol group. The term “thiol group” refers to any organosulfur compound of the form R-SH, where R represents an alkyl or other organic substituent which was designed to react with primary amines. Example thiolation reagents are 2-iminothiolane (2-IT or Traut's Reagent), sodium thioparaconate, γ-thiobutyrolactone and benzoyl homocysteine thiolactone. They are added in a one-step reaction via mechanisms like Schiff'Base formation or ring strain relief. Thiolated organic molecules with a carboxylic acid function can be conjugated to the CLP as well via carbon acid activation with carbodiimides. Interesting are also N-Hydroxysuccinimide activated organic molecules with one or more thiol groups.

More preferably the recombinant bacterial collagen-like protein is functionalized with a thiolactone group. The term “thiolactone group” refers to analogs of lactones in which an oxygen atom is replaced with a sulfur atom and the sulfur atom is within the ring system adjacent to a carbonyl group. A preferred thiolactone is homocysteine thiolactone and its derivatives, most preferred is N-acetylhomocysteine thiolactone.

Compared with traditional radical chain polymerizations known from alkene containing biomaterials like animal derived GelMA or ColMA (methacrylated gelatin and collagen), the addition of a thiol possesses many advantages, such as faster reaction kinetics, superior reaction control, concomitant homogeneity within the resulting network, higher conversion of the functional groups, less shrinkage during crosslinking resulting in less post-polymerization stress due to the highly orthogonal nature of the reaction, the crosslinking reaction is not susceptible to oxygen inhibition, lower radical concentrations (i.e. at least one order of magnitude below chain-growth systems), faster reaction rates as reflected by shorter gel-point times, making them more suitable for cell encapsulation and a better biomimetic matrix (Van Hoorick, Tytgat et al. 2019).

The functionalized recombinant bacterial collagen-like protein according to the invention can be prepared by reacting a recombinant bacterial collagen-like protein as described above with a thiol. Especially suitable molecules for thiolation are N-Hydroxysuccinimide activated organic molecules with one or more sulfhydryl groups, thioesters, 2-iminothiolane (2-IT or Traut's Reagent), sodium thioparaconate, heterocyclic thiolactones such as γ-thiobutyrolactone, N-acetyl homocysteine thiolactone (AcHCT) and benzoyl homocysteine thiolactone.

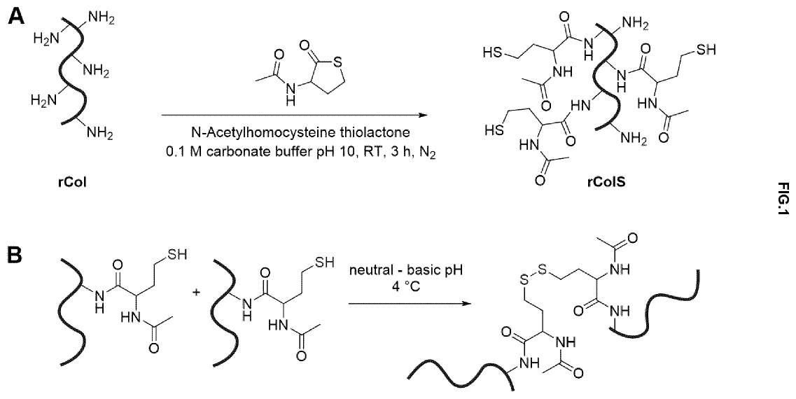

In a preferred embodiment, the functionalized recombinant bacterial collagen-like protein according to the invention can be prepared by reacting a recombinant bacterial collagen-like protein as described above with N-acetyl-homocysteine thiolactone (AcHCT) in an alkaline carbonate buffer. As depicted schematically in FIG. 1, the cyclic thiol ester is conjugated to the primary amine groups of the recombinant bacterial collagen-like protein.

The degree of functionalization of the recombinant bacterial collagen-like protein can be controlled via adjustment of the molar ratio of the recombinant bacterial collagen-like protein and the thiol, like for example N-acetyl-homocysteine thiolactone (AcHCT). Following the functionalization step, the mixture preferably undergoes diafiltration or dialysis to remove the side products. The degree of functionalization can be determined by the known methods such as for example a trinitrobenzene sulfonic acid assay, Ellman Assay, NMR, or HPLC-MS.

The degree of functionalization has an impact on the stiffness of the hydrogel. The higher the degree of functionalization the stiffer the resulting hydrogels. The stiffness can have an impact on protein folding and the size of the pores that are formed in the hydrogel.

Due to the high solubility of the recombinant bacterial collagen-like protein in neutral and alkaline buffers, an unexpectedly high degree of functionalization exceeding 75% can be achieved with minimal compromise to the triple helical protein structure.

In a further embodiment the degree of functionalization with thiol groups of the recombinant bacterial collagen-like protein ranges from 5% to 100% of the sum of primary amines comprising the N-terminal primary amine group and the primary amine groups of the lysine residues of the whole recombinant collagen-like protein.

In a preferred embodiment of the invention the recombinant bacterial collagen-like protein functionalized with thiol groups has a degree of functionalization ranging from 5% to 90% of the sum of primary amines comprising the N-terminal primary amine group and the primary amine groups of the lysine residues of the whole recombinant collagen-like protein.

In a preferred embodiment of the invention the degree of functionalization is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% of the sum of primary amine groups of the recombinant collagen-like protein.

In a preferred embodiment the degree of functionalization is from 10% to 90%, from 20% to 80%, from 25% to 75%, from 30% to 70%, from 40% to 60%.

As described in further detail in Example 1, the degree of functionalization can be modified by adapting the reaction conditions. By tuning the molar ratio of AcHCT to recombinant collagen, thiolated recombinant collagen with different degree of functionalization could be obtained. For example, when the ratio of amine group of recombinant collagen to AcHCT was kept at 1:5, thiolated recombinant collagen with a degree of functionalization around 72% was synthesized.

In comparison, animal-derived collagen is typically only about 10 to 20% substituted as extreme reaction conditions would denature the triple-helical protein structure, producing gelatin which is less mechanically rigid.

A further advantage of the thiolated recombinant bacterial collagen-like protein according to the invention is that it is soluble in water, aqueous buffers at neutral or alkaline pH-value and in particular at physiologically relevant pH-value of 7 to 7.4.

Whilst the underlaying orthogonal step-growth photo-click thiolene chemistry is known in the art, the present invention provides for the first time a thiolated recombinant CLP (rColS) that is sustainably produced and has an animal-free origin.

### Bioinks and Hydrogels

To synthesize collagen hydrogels, different crosslinking methods are known from literature for proteins but only a few are suitable for 3D bioprinting. Herein a light-induced gelation method based on a step-growth orthogonal thiol-ene chemistry is provided. The thiolation of the CLP leads to a new biomaterial (rColS), which favors a fast, signal-induced gelation mechanism without toxic reaction components or side products during or after the gelation.

In a further aspect the present invention therefore relates to a bioink composition comprising
a) from about 0.125 to about 15 wt.-% of at least one functionalized recombinant bacterial collagen-like protein according to the present invention;
b) from about 75 to about 99 wt.-% of aqueous solvent;
c) from about 0 to about 2 wt.-%, preferably about 0.01 to about 2 wt.-%, of at least one photoinitiator;
d) from about 0 to about 5 wt.-% of at least one photocrosslinkable polymer or other photocrosslinkable peptides;
e) from about 0 to about 15 wt.-% of at least one compound selected from additives, rheology modifiers, biopolymers, gelation enhancers, bioactive moieties, peptides, nanocellulose and/or cells;
provided that the sum of all components of the bioink amount to 100 wt.-%.

The aqueous solvent can be water, any aqueous buffer system, such as for example 1×PBS buffer or HEPES buffer, or also a culture medium. Buffer systems and culture mediums are well known in the prior art and can be chosen according to the required application by the skilled person.

In a preferred embodiment the aqueous solvent is water.

In a bioink composition the thiol groups can form polymeric crosslink networks with functionalized recombinant bacterial collagen-like protein molecules or with other components in the bioink composition in the presence of radicals, anions, nucleophiles or combinations thereof.

In one embodiment the bioink composition of present invention therefore further comprises from about 0.125 to about 10 wt.-% of at least one further recombinant bacterial collagen-like protein, wherein said further recombinant bacterial collagen-like protein comprises a functionalization that is different from the functionalization of the functionalized recombinant bacterial collagen-like protein of a).

In a further embodiment of present invention the said at least one further functionalized recombinant bacterial collagen-like protein is a recombinant bacterial collagen-like protein comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1 and wherein the recombinant collagen-like protein is functionalized with at least one alkene group, preferably at least one non-terminal alkene group.

In one embodiment of present invention the said further functionalized recombinant bacterial collagen-like protein is a recombinant bacterial collagen-like protein comprising an amino acid sequence that is at least 60%, preferably at least 70%, more preferably at least 80%, most preferred at least 90% identical to the amino acid sequence of SEQ ID NO:2, SEQ ID NO:3 or SEQ ID NO:4.

In one embodiment of the bioink composition of present invention the degree of functionalization of the said further functionalized recombinant bacterial collagen-like protein ranges from 5% to 100% of the sum of primary amines comprising the N-terminal primary amine group and the primary amine groups of the lysine residues of the whole recombinant collagen-like protein.

In a further embodiment of the bioink composition of present invention the at least one non-terminal alkene group is selected from linear, branched or cyclic non-terminal alkenes, preferably from norbornene or its derivatives, or combinations thereof.

The bioink composition of present invention can contain at least one photoinitiator, preferably a free radical photoinitiator. The amount of photoinitiator added to the bioink composition ranges from 0 to 2 wt.-%, preferably 0.01 to 2 wt.-%, of the total liquid formulation. The photoinitiator(s) are capable of producing radicals when irradiated with actinic radiation.

It is to be understood that the addition of at least one photocrosslinkable polymers or other photocrosslinkable peptides (component d)) into the bioink composition of present invention is optional as the functionalized recombinant bacterial collagen-like protein according to the invention can crosslink with itself. This is a unique feature because the non-functionalized material isn't capable to gel physically.

In one embodiment the crosslinking is an oxidative crosslinking via addition of H₂O₂.

Preferably the photocrosslinkable polymers of component d) are selected from natural (such as hyaluronic acid methacrylate) and synthetic (such as acrylate and methacrylate derivatives of poly(ethylene glycol)) polymers and mixed into the composition to confer additional biological properties (such as anti-fouling) and modulate the physical properties (such as degradability, and swelling behavior in water).

Synthetic peptides with photocrosslinkable groups can be incorporated to confer additional bioactivity such as cell adhesion, stem cell differentiation and enzymatic degradability. Bioactivity broadly describes cell attachment and enzymatic degradation. The bioactivity can be customized via the addition of cell-interactive ligands during the photocrosslinking process.

Photopolymerization or photocrosslinking is preferably triggered by exposure to UV or visible light depending on the photoinitiator chosen. A process for producing a hydrogel by photocrosslinking the functionalized recombinant bacterial collagen-like protein according to the invention also forms part of the invention. Similarly, a process for producing a hydrogel by photocrosslinking the bioink composition according to the invention also forms part of the invention.

In one embodiment of the process of present invention the bioink composition is photocrosslinked using UV light.

In one embodiment of the process of present invention the bioink composition is photocrosslinked using visible light, preferably blue light (380-500 nm). Preferred is light with a wavelength of 390-410 nm, most preferred 405 nm.

The bioink composition can be formulated by dissolving the functionalized recombinant bacterial collagen-like protein according to the invention in an aqueous solution, adding a water-soluble photoinitiator, such as for example lithium phenyl-2,4,6-trimethylbenzoylphosphinate (LAP) or 1-[4-(2-hydroxyethoxy)-phenyl]-2-hydroxy-2-methyl-1-propane-1-one (Irgacure 2959). Other suitable photoinitiators according to present invention are, 2,2′-Azobis [2-methyl-n-(2-hydroxyethyl) propionamid], tris(2,2-bipyridyl) dichlororuthenium(II) hexahydrate, Eosin Y, Ivocerin, ZnTTP and other Irgacure derivates.

Components d) and e) can be optionally included into the composition depending on the end application.

The invention also encompasses a bioink composition comprising from about 1 to 10 wt.-% of the thiolated recombinant bacterial collagen-like protein and/or the recombinant collagen-like protein functionalized with non-terminal alkene, preferably norbornene groups.

The bioink composition according to the invention has lower viscosity compared with formulations using functionalized animal-derived collagen, and therefore puts less shear stress on the cells, and less chances of the nozzle clogging during bioprinting. In the absence of additives, when dissolved in water or physiological buffers, the viscosity of the bioink composition according to the invention can be between 1 to 200 centipoise. In one embodiment the viscosity of the bioink composition is below 120, below 100, below 80, below 60, below 50, below 40, below 30, below 20, below 10 centipoise.

The bioink composition according to the invention can be applied towards the preparation of photocrosslinked hydrogels and sponges. The invention therefore also pertains to a process for producing a hydrogel by crosslinking, preferably photocrosslinking, the functionalized recombinant bacterial collagen-like protein according to the invention. The bioink composition can undergo photocrosslinking to form transparent and colorless hydrogels in the presence of actinic radiation, UV light or visible light (VIS). Here specifically blue light was used.

In a preferred embodiment the light has a wavelength of between 365 nm to 405 nm due to the applied photo initiator LAP.

The resulting pH-value of the bioink composition and thus the hydrogel is preferably between 6.5 and 8. A hydrogel comprising a photocrosslinked functionalized recombinant bacterial collagen-like protein according to the invention therefore is also part of the present invention.

The bioink composition of the present invention can be dispensed using a bioprinter or poured into a mold for photocuring to form hydrogel scaffolds.

Another advantage of the bioink composition according to the invention is that the mechanical properties of the resulting hydrogels are significantly more rigid compared to functionalized animal-derived collagen hydrogels and rigidity (as determined by storage moduli measured using rheology) can be adjusted by varying the concentration of the functionalized recombinant bacterial collagen-like protein according to the invention from 0.125 to 20 wt.-% of the bioink composition and/or by choosing alternative DoFs.

In one embodiment of present invention the bioink composition comprises 0.25 to 15 wt-%, 1.0 to 10 wt-%, 1.5 to 6 wt.-%, more preferably 3 to 6 wt.-% of functionalized recombinant bacterial collagen-like protein according to the invention. In one embodiment the bioink composition comprises only a thiolated recombinant bacterial collagen-like protein according to the invention. In a further embodiment the bioink composition further comprises a recombinant bacterial collagen-like protein functionalized with a non-terminal alkene, preferably a norbornene group according to the invention.

The advantageous mechanical properties of the resulting hydrogels are partly due to the higher solubility of the recombinant collagen itself, enabling the formation of high concentration hydrogels. In comparison, due to solubility considerations, bioinks derived from animal-origin collagen typically have a concentration of less than 0.6%. The rigidity (as determined by storage moduli measured using rheology) of the recombinant collagen hydrogel can be as high as about 6.0 kPa, as exemplified by a 10% hydrogel measured under strain conditions of 0.1%. Hydrogels comprising a photocrosslinked functionalized recombinant bacterial collagen-like protein according to the invention are therefore also part of the invention.

A further advantage of the thiolated recombinant bacterial like protein of present invention is that it allows physical gelation by applying temperature changes. The inventors could show that the base-catalyzed mechanism happened within time when ColS was dissolved at a certain minimum concentration and stored at 4° C. for some time. The gelation was reversible which means that formed gels can be molten with increased temperature and re-gelled when cooled. Further experiments showed that an increase in the degree of substitution yielded in an increased liquification temperature. Yet another aspect of the hydrogel of present invention is that it is injectable (see Example 6). This could be used to design hydrogel formulations which e.g.
a) allow liquification when applied orally (from a gel at RT) or
b) which is initially heated and gels after injection at 37° C.

A further parameter that may be adapted is the concentration of the stock solution. The concentration of the stock solution can have an impact on the gelation properties. Less concentrated stock solutions remain liquid during longer storage times at 4° C., whereas more concentrated stock solutions do not. In one embodiment the stock solution has a concentration of 50 mg/ml, 60 mg/ml, 70 mg/ml, 80 mg/ml, 90 mg/ml, or 100 mg/ml.

Physical gelation of animal-derived collagen is known to literature. The irreversibility of animal-derived collagen is known to literature as well. Increasing the temperature to a too high value leads to irreversible collagen denaturation to gelatin. In contrast, with the recombinant collagen of present invention heating leads to liquification and cooling causes gelation.

The additives that can be incorporated into the bioink composition as component e) include rheology modifiers, gelation enhancers and/or bioactive moieties. These additives can enhance the mechanical, viscoelastic and biological properties of the bioink composition and/or the resulting photocrosslinked hydrogel.

In a further embodiment the bioink composition comprises as component e) glycosaminoglycan, e.g. chondroitin sulfate, hyaluronic acid; silk, elastin, keratin, resilin, titin, elastin-like polypeptides, fibrin, fibrinogen, fibronectin, thrombin, chitosan, carbohydrates like dextran or chitin, growth factors, platelet-rich plasma (PRP), cell binding peptides, oligonucleotides like DNA and RNA.

In a further embodiment the bioink can contain further bioactive components for the usage as injectable scaffolds as dermal filler and for tissue treatments like cartilage, skin or bone repair.

Self-assembling peptides and biopolymers such as nanocellulose can also be incorporated in the bioink composition as component e). Such molecules enhance the gelation and confer additional mechanical properties. Therefore, the invention also pertains to a hydrogel comprising a photocrosslinked functionalized recombinant bacterial collagen-like protein according to the invention the hydrogel further comprising nanocellulose, peptides or mixtures thereof.

Cells can be encapsulated in the photocrosslinking process by adding them into the bioink composition. Cells can also be subsequently incorporated into the photocrosslinked hydrogel scaffolds. Whether printed or cast, a scaffold for tissue engineering comprising the hydrogel above is also subject matter of the present invention.

The resulting photocrosslinked hydrogels support the proliferation of different types of cells in vitro. Cells can advantageously also be incorporated into the bioprinting process. The cell viability, proliferation and spreading in bioprinted hydrogels are significantly better than in bulk-casted hydrogels. Thus, the photocrosslinked hydrogels are especially suited as scaffolds for tissue engineering.

The hydrogels formed from the bioink composition according to the invention, either bioprinted or cast, demonstrate good stability in vivo exceeding three months compared to animal-derived collagen which was resorbed by the natural tissues within a month.

The bioink composition according to the invention can be formulated for different 3D printing or bioprinting technologies, particularly Drop-on-Demand/jetting and digital light printing/stereolithography. The bioink is particularly suited for Drop-on-Demand printing due to its low viscosity.

The bioink is also suited towards digital light printing, where bioink compositions containing 0.5 to 6 wt.-% of functionalized recombinant bacterial collagen-like protein according to the invention have been successfully printed into 3D hydrogel constructs.

Preferably bioink compositions containing 1.5 to 6 wt.-% of functionalized recombinant bacterial collagen-like protein according to the invention, more preferably 3 to 6 wt.-% can be printed into 3D hydrogel constructs.

In a further aspect the present invention relates to a process for producing a hydrogel by photocrosslinking the functionalized recombinant bacterial collagen-like protein or the bioink composition as described herein.

In yet a further aspect the present invention relates to a hydrogel obtained by the process according to the present invention.

In one embodiment of the hydrogel of present invention the hydrogel further comprises nanocellulose, peptides or mixtures thereof.

In yet a further aspect the present invention relates to a scaffold for tissue engineering comprising the hydrogel as described herein.

The use of the word “a” or “an” may mean “one,” but it is also consistent with the meaning of “one or more,” “at least one,” and “one or more than one”. The use of the term “another” may also refer to one or more. The use of the term “or” in the claims is used to mean “and/or” unless explicitly indicated to refer to alternatives only or the alternatives are mutually exclusive.

“At least one”, as used herein in relation to any component, refers to the number of chemically different molecules or groups, i.e., to the number of different types of the referenced species, but not to the total number of molecules or groups in the composition or compound. For example, “at least one thiol group” means that at least one type of thiol groups is used but that also two or more different types of thiol groups can be present but does not mean that only one or more groups (number) of thiol groups are present.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. The term “comprises” also encompasses and expressly discloses the terms “consists of” and “consists essentially of”. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “around”, “approximately” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by ±1, 2, 3, 4, 5, 6, 7, 8, 9, or 10%. Accordingly, the term “about” may mean the indicated value±5% of its value, preferably the indicated value±2% of its value, most preferably the term “about” means exactly the indicated value (±0%).

The following examples serve to illustrate the present invention and should not be construed as limiting the scope thereof.

### EXAMPLES

### Example 1: Preparation of Thiolated Recombinant Collagen-Like Protein (rColS) with Different Degrees of Functionalization (DoF)

The functionalized recombinant CLP (rCol) according to the invention was prepared as follows:

A clear 4% (w/V) rCol solution in 0.1 M carbonate buffer (pH 10) was prepared at RT (20° C.). The solution was transferred into a 2-neck flask together with 1 mM EDTA to avoid thiol oxidation by metal ions. The solution was degassed and flushed with N₂ (3× times each; 1-3 min/step). 160.3 mg/ml DL-N-Acetyl homocysteine thiolactone (short: AcHCT; Sigma&Aldrich; article number: A16602-25G) was dissolved in degassed 0.1 M carbonate buffer (pH 10) with 160.3 mg/ml and added via syringe and septum. After stirring for 3 h at RT (20-25° C.), the reaction mixture was dialyzed for 24 h at RT against degassed ddH₂O (cut-off: 3.5 kDa). Water was changed four times by new degassed water. 1 mM EDTA was added to the first two washing solutions. The purified product was freeze dried to generate a white sponge-like material which was stored under N₂ at −80° C. for further usage. The DoF was quantified via Ellman Assay and ¹H-NMR.

Carbonate buffer: Mix 60 ml 0.1 M Na₂CO₃ solution with 40 mL 0.1 M NaHCO₃ solution.

TABLE 1
DoF of different rColS synthesis trials

| Equivalents | DoF (average signal of |
|---|---|
| NH_{2(rCol)}:AcHCT | Ellman Assay and ¹H NMR) |
| 1:0 | 0% |
| 1:0.25 | 15% |
| 1:0.5 | 22% |
| 1:1 | 39% |
| 1:2 | 55% |
| 1:5 | 72% |

### Example 2: Material Safety-Cell Viability of Dissolved and Modified CLP

The CellTiter-Glo® Luminescent Cell Viability-Assay was performed to determine cytotoxic effects of the synthesized collagen derivatives in alignment with the manufacturer protocol. 3T3 mouse fibroblast cells were pre-seeded with 1×10⁴ cells/well (1×10⁵ cells/ml, 0.1 ml) into a white 96 well plate for luminescence measurements.

10 mg/ml sample stock solutions in culture medium were diluted with culture medium for the following concentrations: 10, 5, 1, 0.1 mg/ml. After 14-24 h incubation under culture conditions, the cell medium was replaced by 100 μl test solution. Each formulation was tested in triplicate. As background, the formulation was tested without cells with 100 μl each. Fresh medium with cells was used for the negative control. Cells were exposed to the test formulations for 24 h under cell culture conditions. Then, the CellTiter-Glo® reagent was prepared (1:1 mixture of CellTiter-Glo® substrate and CellTiter-Glo® buffer) and equilibrated to RT (20-25° C.) for 30 min. The reagent was added with 100 μl/well. The plate was orbitally stirred for 2 min using a TecanReader Pro200 followed by 10 min incubation time. Next, the luminescence was measured with an integration time of 0.1 seconds per well. The average signal of the positive control was subtracted from each test sample and the sample signals were normalized on the negative control. The average of the sample values as well as the standard deviation was calculated.

### Example 3: Bioink Preparation

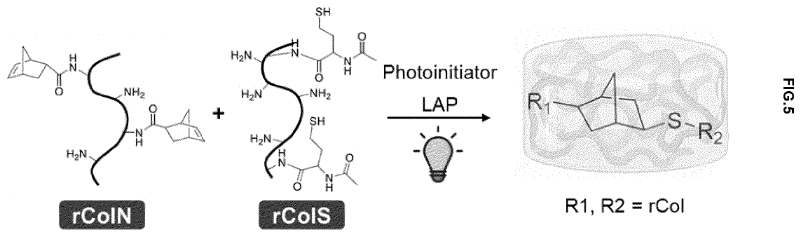

The bioink according to the invention was formulated by dissolving the functionalized recombinant bacterial collagen-like protein from Example 1 in phosphate-buffered saline, as well as rColN as described herein above. Both homogenous solutions were mixed and spiked with fully dissolved lithium phenyl-2,4,6-trimethylbenzoylphosphinate (LAP) in phosphate-buffered saline. The final concentration of LAP was 0.03% (w/v) of the bioink composition (see schematic FIG. 5). Optionally, cells can be added to the formulation as well. The formulation was exposed to light of a wavelength of 365-405 nm to induce gelation. Depending on the DoF and the applied concentrations of rColN, rColS and LAP, different irradiation times were needed. The following table shows tested formulations regarding hydrogel formation. rColN and rColS were applied with identical mass ratios per formulation and different DoF. The DoF were selected to be similar.

TABLE 2
Gelation trials with rColN, ColS and LAP. rColN and ColS were always
used with identical concentrations. A LAP concentration of 0.03%
was used for all formulations. 1x PBS was used as solvent.

| Bioink | DoF | DoF | [total Collagen] (mg/ml) (1:1 mixture of | | | | |
|---|---|---|---|---|---|---|---|
| Classifi- | (rColN) | (rColS) | rColN and rColS) | | | | |
| cation | (%) | (%) | 2.5 | 5.0 | 10 | 20 | 40 |
| Extra low | 8 | 8 | X | X | X | X | ✓ |
| Extra low - | 12 | 17 | X | X | X | ✓ | ✓ |
| low | | | | | | | |
| Low | 29 | 32 | X | ✓ | ✓ | ✓ | ✓ |
| Medium | 41 | 45 | ✓ | ✓ | ✓ | ✓ | ✓ |
| Medium | 48 | 45 | ✓ | ✓ | ✓ | ✓ | ✓ |
| high | 80 | 81 | ✓ | ✓ | ✓ | ✓ | ✓ |

All samples were irradiated with the same intensity. Successful gelation (✓) and insufficient gelation (X) was documented.

### Example 4: Material Suitability for Cell Cultivation of rColN/rColS Hydrogels

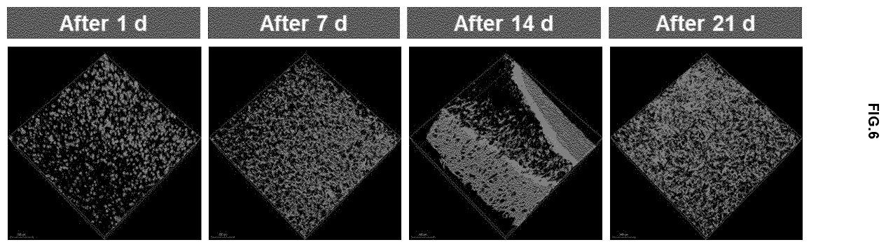

A formulation of 2.5 mg/ml rColN (48% DoF), 2.5 mg/ml rColS (32% DoF) and 0.3 mg/ml LAP (lithium phenyl-2,4,6-trimethylbenzoylphosphinate) was prepared in 1×PBS buffer together with 2.5×10⁶ HDF cells/ml (human dermal fibroblasts). Respectively 200 μl sample/well was filled in a sterile 8 well chamber slide. The formulation was irradiated with light of a wavelength area of 365 nm-405 nm until the hydrogel solidified. Each sample was mounted with 200 μl culture medium and incubate for 3 weeks. The medium was exchanged after 2 h, after 24 h and then every 2-3 days. After 1, 7, 14 and 21 days, a LIVE/DEAD staining (ThermoFisher Scientific) was used to determine viable (green) and dead (red) cells (FIG. 6). After 21 d, also a depth coding was made.

### Example 5: 3D Bioprinting

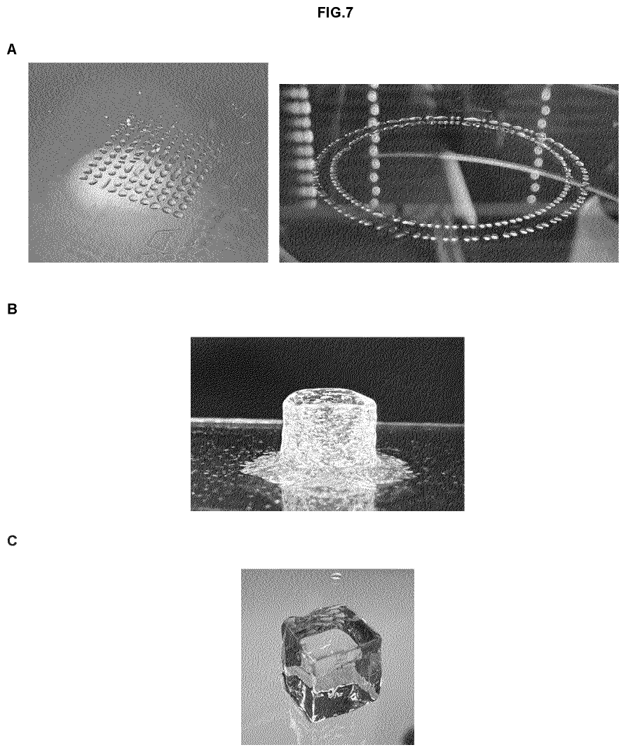

To show the printability of the material a drop-on demand printer was used. 10 mg/ml rColN (DoF of 59%) and 10 mg/ml rColS (DoF of 48%) were dissolved in 1×PBS (phosphate buffered saline) together with 0.03% LAP. The homogenized mixture was added to the machine and droplets were printed in different patterns. After light irradiation, the droplets were gelled (FIG. 7).

### Example 6: Physical Gelation

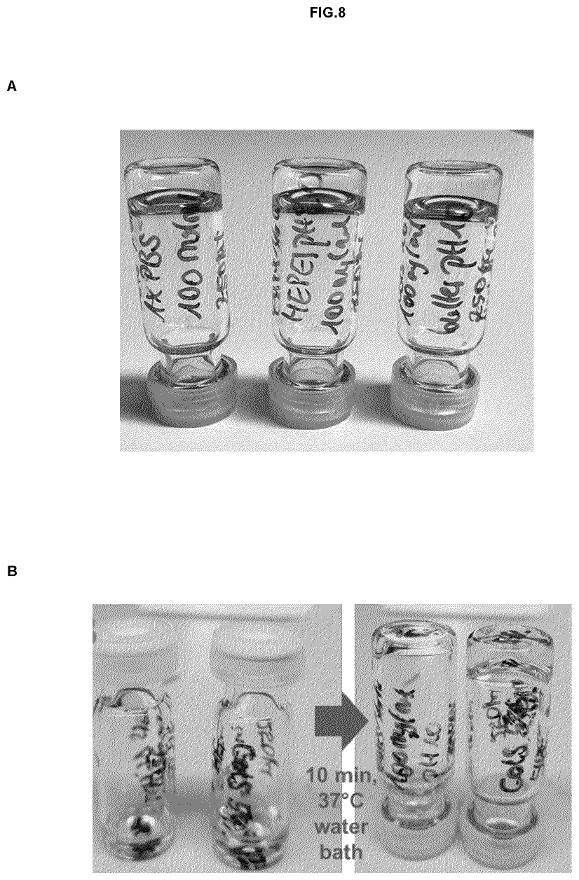

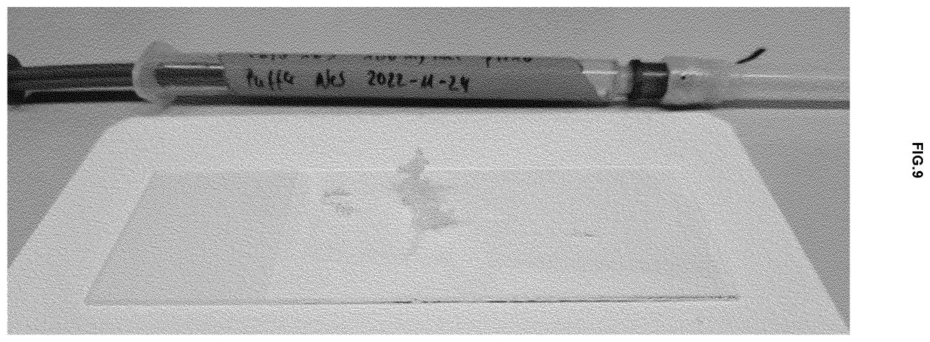

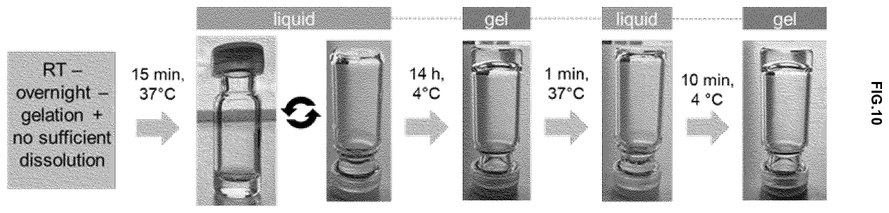

100 mg/ml ColS solutions were prepared in 3 different buffer systems (1×PBS, 0.1 M HEPES buffer pH-value 8.0, carbonate buffer pH-value 10). After complete dissolution, the formulations were transferred in 2 ml HPLC glass vials and incubated for 24 h at 4° C. Then the vials were turned upside down to prove gelation. The DoF (degree of functionalization) influences the liquification temperature. While a ColS hydrogel of 16% DS liquified at 37° C. and identical hydrogel with a DS of 54% didn't (FIG. 8). To show injectability a liquid 100 mg/ml ColS formulation was filled into a 1 ml syringe and after incubation at 4° C. for 24 h, the formulation was ejected by a 26G needle (FIG. 9). Finally, the reversibility of gelation was shown by taking 100 mg/ml ColS solution (DS of 16%) and performing adjacent gelation and liquification cycles (FIG. 10).

### Example 7: Viscosity Measurements

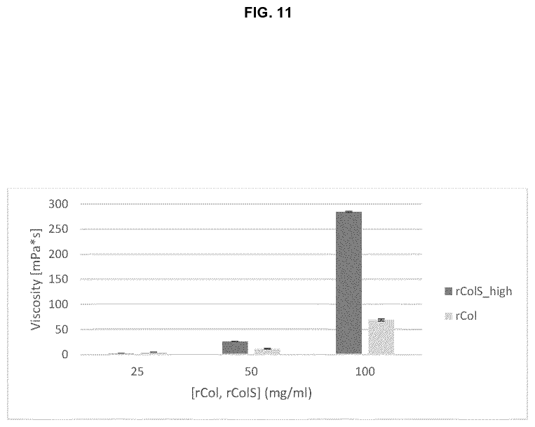

To better understand the viscosity of the material, rheological measurements were performed with the unmodified rCol. Solutions viscosities were measured using an Anton-Paar MCR 502 WESP system equipped with a plate-cone extension. The lower plate was flat (stainless steel, Ø 50 mm, CP50) while the cupper cone had a 1° angle to the middle (stainless steel, Ø 50 mm, CP50-1) with a truncation of 99 μm. A standard flow curve program was applied. The measurement sequence is described in the following. A sample load of 750 μl was used. The stock solution was made one day in advance and left for complete dissolution on an orbital shaker at 20° C. overnight. Excess sample was removed with a tissue. The measurement was performed at 20° C. data points and a shear of 0.1-1000. Data extraction: The average of data points in the linear range was calculated for each measurement and the average of three subsequent measurements was calculated along the standard deviation. The results of the viscosity measurements are depicted in FIG. 11.

### Example 8: Rheological Measurements

Rheological measurements were performed with the benchtop ElastoSens™ Bio device from Rheolution Live Sciences. The non-destructive non-contact measurement relies on induced vibration of the sample holder silicon bottom whose amplitude is recoded by a laser. The stiffer the formulation, the lesser the response in amplitude.

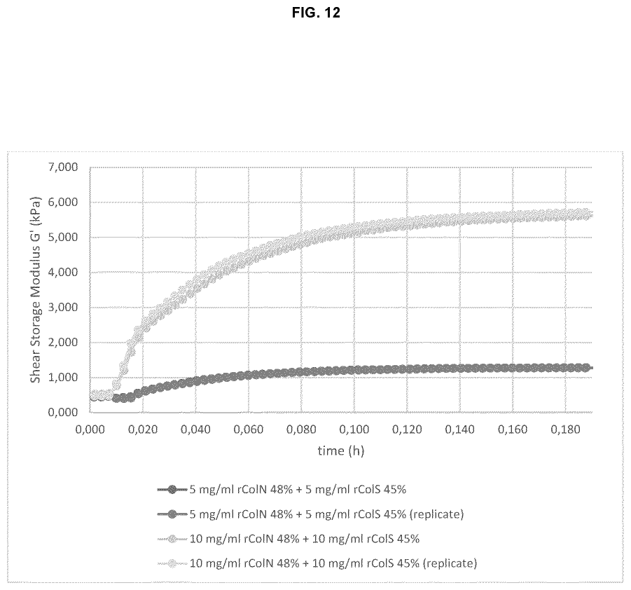

For photopolymerization, hydrogel formulation with 2.2 ml were prepared. 2 ml was transferred into the calibrated sample holder using reverse pipetting. The sample was irradiated with 405 nm (50% lamp power; equals a light power of 11.6 mW/cm² according to the providers light power chart) until a stable Shear Storage Modulus (G′). The stiffness was measured every 10 seconds. The hydrogel height was recorded by the device ensuring no significant decrease. If not stated otherwise, the reaction temperature was kept constant at 25° C. If the recorded Shear Storage modulus (G′) was below 500 Pa using the standard stiff mode, the measurement was repeated with 7 ml of the same formulation according to the user manual in soft mode. The results can be seen in FIG. 12.

1. Recombinant bacterial collagen-like protein, preferably comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1, wherein the recombinant collagen-like protein is functionalized with at least one thiol group.

2. The recombinant bacterial collagen-like protein according to claim 1, wherein the amino acid sequence is at least 60%, preferably at least 70%, more preferably at least 80%, most preferred at least 90% identical to the amino acid sequence of SEQ ID NO:2, SEQ ID NO:3 or SEQ ID NO:4.

3. The recombinant bacterial collagen-like protein according to any one of claims 1 or 2, wherein the degree of functionalization with thiol groups is from 5% to 100% of the sum of primary amines comprising the N-terminal primary amine group and primary amine groups of lysine residues of the recombinant collagen-like protein.

4. Bioink composition comprising:
a) from about 0.125 to about 15 wt.-% of at least one functionalized recombinant bacterial collagen-like protein according to any one of claims 1 to 3;
b) from about 75 to 99 wt.-% of an aqueous solvent;
c) from about 0 to 2 wt.-% of at least one photoinitiator;
d) from about 0 to 5 wt.- % of at least one photocrosslinkable polymer or other photocrosslinkable peptides;
e) from about 0 to about 15 wt.-% of at least one compound selected from additives, rheology modifiers, biopolymers, gelation enhancers, bioactive moieties, peptide, nanocellulose and/or cells;
provided that the sum of all components of the bioink amounts to 100 wt.-%.

5. The bioink composition according to claim 4, where the formulation comprises from about 0.125 to about 10 wt.-% of at least one further recombinant bacterial collagen-like protein, wherein said further recombinant bacterial collagen-like protein comprises a functionalization that is different from the functionalization of the functionalized recombinant bacterial collagen-like protein of a).

6. The bioink composition according to claim 5, wherein the said at least one further functionalized recombinant bacterial collagen-like protein is a recombinant bacterial collagen-like protein comprising an amino acid sequence that is at least 60% identical to the amino acid sequence of SEQ ID NO:1, wherein the amino acid sequence comprises a deletion of at least 38 amino acids at the N-terminus of the amino acid sequence of SEQ ID NO:1 and wherein the recombinant collagen-like protein is functionalized with at least one alkene group.

7. The bioink composition according to claim 5, wherein the said at least one further functionalized recombinant bacterial collagen-like protein is a recombinant bacterial collagen-like protein comprising an amino acid sequence that is at least 60%, preferably at least 70%, more preferably at least 80% , most preferred at least 90% identical to the amino acid sequence of SEQ ID NO:2, SEQ ID NO:3 or SEQ ID NO:4.

8. The bioink composition according to any one of claims 4 to 7, wherein the degree of functionalization of the said further functionalized recombinant bacterial collagen-like protein ranges from 5% to 100% of the sum of primary amines comprising the N-terminal primary amine group and the primary amine groups of the lysine residues of the recombinant collagen-like protein.

9. The bioink composition according to claims 4 to 8, wherein the at least one alkene group is a non-terminal alkene group selected from linear, branched or cyclic non-terminal alkenes, preferably from norbornene or its derivatives, or combinations thereof.

10. Process for producing a hydrogel by crosslinking, preferably photocrosslinking, the functionalized recombinant bacterial collagen-like protein according to any one of claims 1 to 3 or the bioink composition according to any on of claims 4 to 9.

11. The process according to claim 10, characterized in that the bioink composition is photocrosslinked using UV light or visible light, preferably blue light.

12. Hydrogel obtained by the process of claim 10 or 11.

13. The hydrogel according to claim 12, wherein the hydrogel further comprises nanocellulose, peptides or mixture thereof.

14. A scaffold for tissue engineering or tissue regeneration comprising the hydrogel according to claims 12 or 13.
